(19) United States
(12) Patent Application Publication
(10) Pub. No.: US 2026/0302267 A1
(43) Pub. Date: Oct. 1, 2026

(54) Phenolic Coatings for Enhanced Resistance to Hydrocarbon Poisoning of Proton Exchange Membrane Fuel Cell Electrodes

(71) Applicant: University of Cincinnati, Cincinnati, OH (US)

(72) Inventor: Anastasios Angelopoulos, Loveland, OH (US)

(73) Assignee: University of Cincinnati, Cincinnati, OH (US)

(21) Appl. No.: 19/480,204

(22) PCT Filed: May 2, 2024
(86) PCT No.: PCT/US2024/027496

§ 371 Date: Oct. 30, 2025

Related U.S. Application Data
(60) Provisional application No. 63/463,519, filed on May 2, 2023.

(51) Int. Cl. H01M 4/92

(52) U.S. Cl. CPC H01M 4/926

(57) ABSTRACT

An electrocatalyst ink composition is provided. The ink composition includes a liquid vehicle, particles with at least one electrocatalyst metal, and at least one compound having a phenolic moiety. In various embodiments, the compound comprising a phenolic moiety is resorcinol and the electrocatalyst metal is a platinum-bismuth alloy.

### CROSS REFERENCE TO RELATED APPLICATIONS

This application is a continuation of PCT Application No. PCT/US24/24692 filed Apr. 15, 2024, which claims the benefit of the filing date of U.S. Provisional Application Ser. No. 63/463,519, filed May 2, 2023, which applications are hereby incorporated by reference in their entirety.

### TECHNICAL FIELD

The present invention relates to electrocatalyst inks for fuel cell electrodes.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

There is growing interest in the use of reformed hydrocarbon waste as a zero-carbon emission source of power in low temperature proton exchange membrane fuel cells (PEMFC). As in the case of petroleum-based fuels such as methanol and dimethyl ether, as well as biofuels such as ethanol, the key limitation to their utilization is the presence of carbon monoxide (CO). CO is present in the original feed or is formed as a byproduct of hydrocarbon oxidation and, at sufficiently low temperatures, is known to irreversibly bind to the precious metal surface of neat, alloyed, or mixed-metal electrodes. Such bonding can prevent reaction of the excess H₂ in the fuel. Examples include pure Pt or Pd nanoparticles supported on carbon or Pt or Pd alloys or mixed metals with Ru, Bi, Co, or Ni. In the case of the pure precious metals, CO concentrations as low as 10 ppm are sufficient to degrade performance. The introduction of alloys can retain performance at these concentrations. However, reformate typically contains CO concentrations of between 1% to more than 10%, depending on the raw material feed. Water-gas shift (WGS) reactors as well as pressure swing adsorption processes must be employed subsequent to reforming to reduce CO concentration to the ppm levels required in current state-of-the art low temperature PEMFCs due to electrocatalyst poisoning. For the 1% CO concentration typically obtained from methane steam reforming, the cost of high purity H₂ can be more than 5 times that of the untreated syngas. Although high temperature phosphoric acid fuel cells (PAFCs) operating at 160-180° C. can use this syngas directly, CO concentrations obtained from the reformate product of waste streams can exceed 10% and are unsuitable in even these systems.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to an electrocatalyst ink composition. The ink composition includes a liquid vehicle, particles with at least one electrocatalyst metal, and at least one compound having a phenolic moiety.

In one embodiment, the compound comprising a phenolic moiety is selected from the group consisting of resorcinol, phenol, catechol, hydroquinone, phloroglucinol, cresol, halophenol, aminophenol, hydroxybenzoic acid, vannilic acid, vanillin, ferulic acid, benzoic acid, tiglic acid, and dihydroxybiphenyl. In another embodiment, the compound comprising a phenolic moiety is resorcinol.

In one embodiment, the electrocatalyst metal is selected from the group consisting of carbon, palladium, ruthenium, rhodium, gold, copper, cobalt, nickel, and the rare earth elements. In another embodiment, the electrocatalyst metal comprises a Platinum-Bismuth alloy.

In one embodiment, the particles are in a form selected from the group consisting of nanoparticles, pellets, platelets and combinations thereof. In another embodiment, the particles are in the form of nanoparticles. In one embodiment, the nanoparticles are attached to a support. In another embodiment, the support comprises carbon.

Another aspect of the present invention is directed to a method for the fabrication of an electrocatalyst layer. The method involves depositing an ink composition onto a substrate. The ink composition includes a liquid vehicle, particles having at least one electrocatalyst metal, and at least one compound with a phenolic moiety. In one embodiment, the substrate includes a proton exchange polymer. In another embodiment, the proton exchange polymer is Nafion.

### BRIEF DESCRIPTION OF THE DRAWINGS

The objects and advantages of the disclosed invention will be further appreciated in light of the following detailed descriptions and drawings in which:

FIG. 1 is a graph showing chronopotentiometry data generated for 5 cm² fuel cell operating with an air cathode and 1% CO in hydrogen anode at a temperature of 60° C., a relative humidity of 90%, and a stoic of 6 on both anode and cathode. Time zero indicates when anode flow was switched from pure hydrogen to 1% CO. Pt loading was 1 mg/cm² for all catalysts. X-axis indicates time in seconds.

FIG. 2A is a graph showing a comparison of permeability at 1600 rpm in O₂ saturated 0.1M HClO₄ for two electrodes. Isolated points indicate result with resorcinol.

FIG. 2B is a graph showing a comparison of limiting current at 1600 rpm in O₂ saturated 0.1M HClO₄ for two electrodes. Isolated points indicate result with resorcinol.

FIG. 2C is a graph showing a comparison of kinetic current at 0.9 V vs. RHE at 1600 rpm in O₂ saturated 0.1M HClO₄ for two electrodes. Isolated points indicate result with resorcinol.

FIG. 2D is a graph showing a comparison of half-wave potential at 1600 rpm in O₂ saturated 0.1M HClO₄ for two electrodes. Isolated points indicate result with resorcinol.

FIG. 3A is a graph showing cyclic voltammetry curves of PtBi NP electrodes in N₂ saturated 0.5 M H₂SO₄ with different coatings.

FIG. 3B is a graph showing cyclic voltammetry curves of PtBi NP electrodes in 0.1M HClO₄ with different coatings.

FIG. 4 is a graph showing cell potential over time for a PtBi catalyst with resorcinol (upper line) vs. a PtBi catalyst without resorcinol (middle line) vs. a Pt catalyst without resorcinol (bottom line).

### DEFINITIONS

As used herein, the term “electrocatalyst” means a catalyst that when incorporated into an electrode, such as a gas diffusion electrode, facilitates an electrochemical reaction. The particular electrocatalyst metal(s) employed will depend on the intended use of the ink but, for fuel cell use, preferred electrocatalyst metals include the platinum group metals and noble metals, particularly Pt, Ag, Pd, Ru, Os and their alloys. The metal phase can also include a metal selected from the group Ni, Rh, Ir, Co, Cr, Mo, W, V, Nb, Al, Ta, Ti, Zr, Hf, Zn, Fe, Cu, Ga, In, Si, Ge, Sn, Y, La, lanthanide metals and combinations or alloys of these metals. Preferred metal alloys include alloys of Pt with other metals, such as Ru, Os, Cr, Ni, Mn and Co.

As used herein, a “liquid vehicle” is a liquid formulation that is normally aqueous based, by which is meant that the vehicle typically comprises at least 50 weight % water. The aqueous vehicle can, however, also contain water miscible solvents, such as alcohols or ketones, to alter the viscosity of the ink and/or to provide additional properties, such as to act as a humectant. Examples of suitable alcohols include isopropanol and ethylene glycol. Organic solvent based systems such as those containing acetone or pyrrolidinone can also be used.

As used herein, “Nafion” refers to the trademarked material sold by DuPont-polysulphonic tetrafluoroethylene. Nafion is composed of a tetrafluoroethylene backbone with side chains terminated with a sulphonic acid group. The sulphonic acid group is the active group of the ionomer, providing the mechanism for the conduction of protons to the cathode.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The compositions and methods of the present disclosure can comprise, consist of, or consist essentially of the essential elements and limitations of the disclosure described herein, as well as any additional or optional ingredients, components, or limitations described herein or otherwise useful.

All percentages, parts and ratios herein are based upon the total weight of the compositions of the present disclosure, unless otherwise indicated.

As used herein, the term “about,” when referring to a value or to an amount of mass, weight, time, volume, pH, size, concentration or percentage is meant to encompass variations of in some embodiments+20%, in some embodiments+10%, in some embodiments+5%, in some embodiments+1%, in some embodiments+0.5%, and in some embodiments+0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present invention involves the discovery that the addition of phenolic compounds such as resorcinol to existing catalyst ink formulations can substantially mitigate the time it takes for CO to poison a precious metal catalyst surface. In the case of an electrocatalyst formulated to oxidize CO, this invention can permit sufficient time for the oxidation rate to prevent complete loss of electrocatalytic activity.

A first aspect of the present invention provides an electrocatalyst ink comprising a liquid vehicle, particles including an electrocatalyst metal and a compound comprising a phenolic moiety. Phenolic compounds or phenols are preferably compounds which consist of an aromatic ring (arene) and one or more hydroxy groups bonded to it. In one embodiment, the phenolic compound is selected from the group consisting of resorcinol, phenol, catechol, hydroquinone, phloroglucinol, cresol, halophenol, aminophenol, hydroxybenzoic acid, vannilic acid, vanillin, ferulic acid, benzoic acid, tiglic acid, and dihydroxybiphenyl. The phenolic compound resorcinol has proven to be particularly suitable and reliable for use in the present invention.

In one embodiment, the electrocatalyst material comprises platinum and bismuth. Other useful electrocatalyst materials include carbon, palladium, ruthenium, rhodium, gold, copper, cobalt, nickel, and the rare earth elements. The particles of the present invention are typically used in nanoparticle form to maximize their surface to volume ratio. These nanoparticles be used either attached to a support such as carbon or on unsupported (free-standing) form. In addition to these nanopowders, other particle forms include larger pellets or platelets.

The present invention may be used in a fuel cell. In various embodiments, the fuel cell is a hydrogen, methanol, ethanol, or dimethyl ether fuel cell.

In one embodiment, the fuel cell comprises an electrolyte that is a proton exchange membrane (PEM). PEM's are very highly acidic materials which do not conduct electrons but are good conductors of protons. Such fuel cells rely on the ready availability of protons generated at the anode, which pass through the PEM, to react with electrons at the cathode. Accordingly, these fuel cells best operate at highly acidic pH, meaning that there is a plentiful supply of protons or hydrogen ions.

A proton exchange polymer is a polymer which readily transports protons along the polymer, but which is relatively resistant to the passage of anions and electrons. Typically, a proton exchange polymer permits the passage of protons at least 10 times more readily than it permits the passage of similarly sized anions. Preferably a proton exchange polymer will permit the passage of protons at least 50, or more preferably at least 100 times, more readily than the passage of similarly sized anions.

The proton exchange membrane (PEM) is a mature technology including now several different proton exchange chemistries. In one embodiment of the present invention, Nafion is used. Alternatively, polybenzimidazole (PBI) can be used at higher temperatures and lower humidity levels than Nafion, e.g. numerous examples of the PBI class of proton exchange membranes are described in Li et al, Fuel Cells, Vol 4, Issue 3, pp 147-159, August 2004.

Other proton/cation conducting membranes exist. For example, proton conducting membranes, especially with hydrocarbon backbone chemistry, are described in Rikukawa M and Sanui K, Prog. Polym. Sci. 25(2000) 1463-1502. These membranes are typically thinner than their Nafion counterparts, and they possess greater material strength. New quaternary ammonium-biphosphate ion-coordinated polymer (ion-pair) electrolytes have also been developed to mitigate the phosphoric acid loss in the presence of water that is observed utilizing standard phosphoric acid-doped polybenzimidazole (PBI) membranes. (Lim, K. H., et al., ACS Energy Letters, 2022: p. 529-536.)

The ion-pair electrode technology of the previous paragraph has also been coupled with polymer composite inks which employ blends of perfluorosulfonic acid (PFSA) and poly tetrafluorostyrene-phosphonic acid (PWN) polymers to achieve high proton transfer rates. (Lim, K. H., et al., Nature Energy, 2022. 7(3): p. 248-259)

Important characteristics for the proton conducting membranes are its conductivity and thickness. The preferred membrane for use in the various aspects of the invention consists of a film of thickness of at least 10 microns and preferably less than 200 microns. A preferred membrane thickness will typically be in the range of 15-100 microns and typical Nafion membrane thickness for hydrogen fuel are on the order of 40-60 microns and on the order of 150 microns for methanol fuel. Often hydrocarbon based membranes will be thinner than their Nafion counterparts on the order of 20-40 microns for either hydrogen or methanol fuel. The proton conductivity of the membrane is preferably greater than 100 mS/cm².

### EXAMPLES

### Example 1

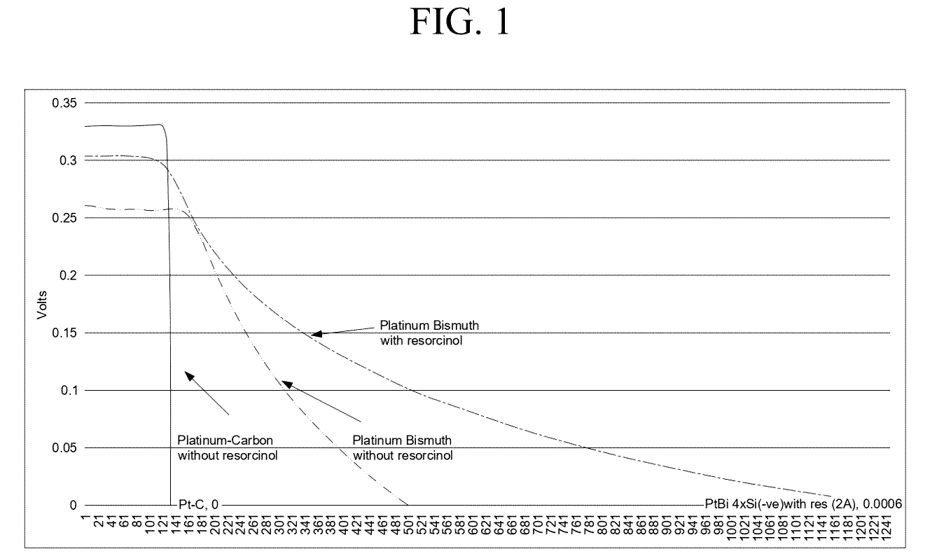

To prepare ink for an electrocatalyst loading of 1 mg Pt from chemically de-alloyed non-carbon supported Platinum-Bismuth nanoparticle powder, 1 ml of a 70/30 IPA/water solution is added to 1.2 mg of the catalyst powder and sonicated to disperse. To this is added 1 mg of Nafion and a silica nanoparticle wetting agent and sonicated. This constitutes a standard make-up procedure for an electrocatalyst known to oxidize CO more rapidly than pure Pt. By contrast, a standard make-up procedure for 50% pure Pt by weight on a carbon-support uses 2 mg of catalyst powder for a 1 mg coating (with the same amount of IPA/water and Nafion as before). In one embodiment of the present invention, 10-3 mg of resorcinol is added to either of the catalyst inks. The result of resorcinol addition to the Platinum-Bismuth alloy ink at a loading of 1 mg/cm² is shown by chronopotentiometry data in FIG. 1.

The data of FIG. 1 was generated for a 5 cm² fuel cell operating with an air cathode and 1% CO in hydrogen anode at a temperature of 60° C., a relative humidity of 90%, and a stoic of 6 on both anode and cathode. Time zero indicates when anode flow was switched from pure hydrogen to 1% CO. A substantial increase in the time it takes for CO to completely cover the surface and loss of voltage at a stable operating current of 2 A (0.4 A/cm²) was observed. Polarization performance of the electrode recovers after the anode stream is returned to pure hydrogen.

### Example 2

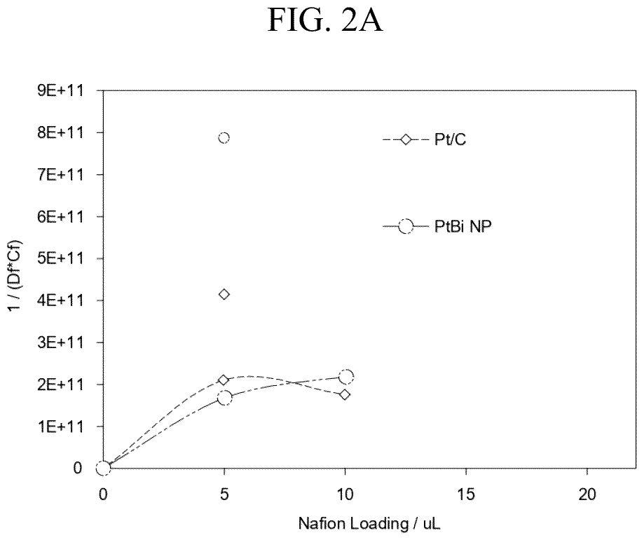

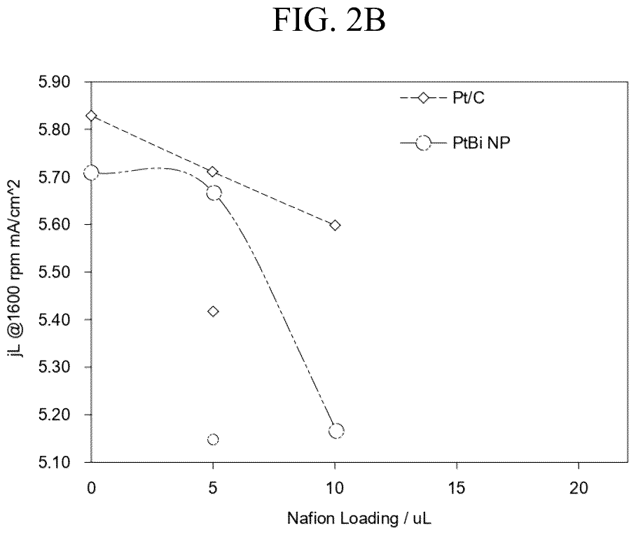

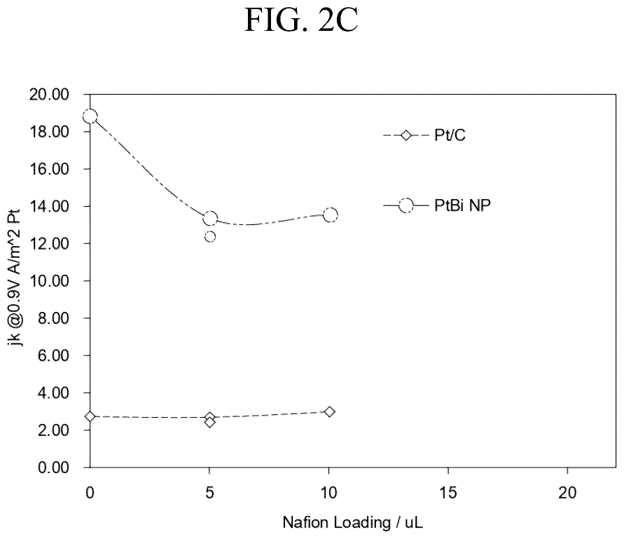

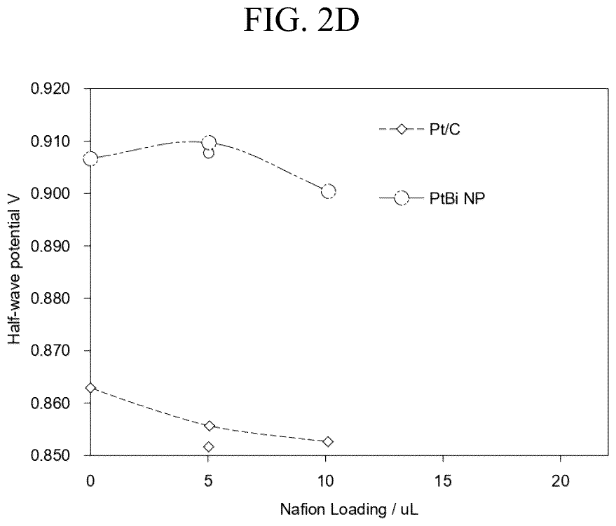

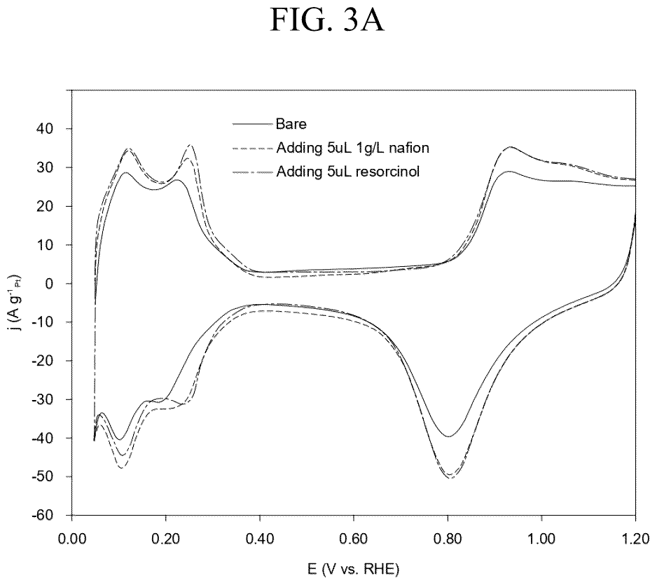

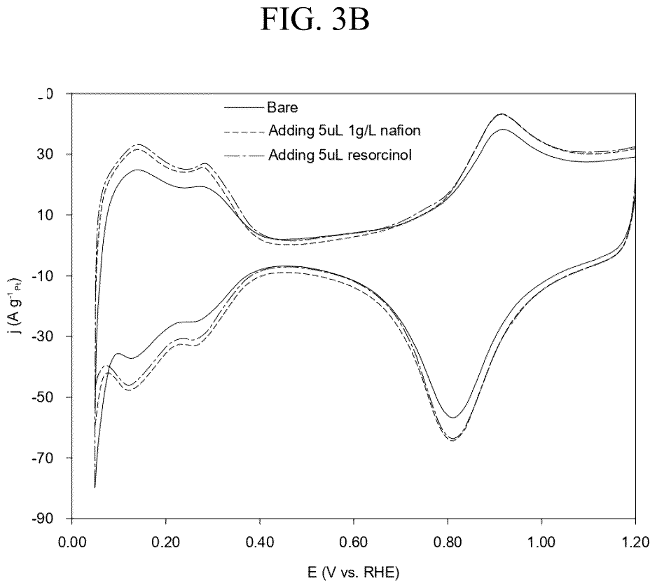

As part of the development of the present invention, it was determined that the source of enhanced CO tolerance is increased resistance to molecular diffusion through the Nafion layer that coats the Pt surface. This was established utilizing oxygen as a probe molecule in rotating disk electrode measurements of the permeability coefficient (product of the diffusion and solubility coefficients) through a Nafion layer. The data shown in FIG. 2A compares the inverse of the permeability coefficient for various electrode surfaces both as a function of both the amount of Nafion added and the presence of resorcinol. The amount of Pt catalyst present on the surface of these small electrodes is 5 micrograms. The x axis represents the microliters of 1 gram/liter Nafion solution added to the ink. Hence, 5 microliters represents an equivalent weight as that of Platinum. It was observed that the addition of resorcinol has the most pronounced effect on the platinum bismuth electrode, substantially reducing the permeability coefficient with a concomitant impact of the diffusion limited current shown in FIG. 2B. By contrast, resorcinol has no impact on the reaction-kinetics limited current and half-wave potential shown in FIGS. 2C and 2D. Given the small relative size of the H₂ molecule, there is no impact on the cyclic voltammetry behavior in the hydrogen underpotential deposition region (below 0.4 V) shown in FIG. 3. On the other hand, the size of the CO molecule relative to that of O₂ is expected to encounter comparable mass transport resistance in Nafion.

Without being bound by theory, the reason why such resistance to molecular transport mitigates poisoning appears to be due to the highly exothermic nature of CO adsorption on Pt. The rate of CO adsorption is limited by the rate of CO transport from the gas phase to the surface, which we can denote as k_{MT}. This rate constant is dependent on the gas phase CO partial pressure, the permeability coefficient of CO through the Nafion surface layer, and the thickness of this layer. Removal of CO from the surface relies on oxidation at the appropriate anodic potential and is known to be limited by reaction with hydroxyl groups in close proximity. If the rate of this oxidation is denoted as k_{OX} and CO adsorption is assumed to not compete with other species due to its highly exothermic nature, then the number of Pt surface sites that are not poisoned can be written simply by the ratio: k_{OX}/(k_{MT}+k_{OX}). The present invention employs phenolic additives to reduce k_{MT} so as to drive this ratio closer to 1. Such behavior has not previously been described in the published literature. While the ability of Nafion to irreversibly bind resorcinol has been previously disclosed, the capacity for resorcinol to impede the mass transport resistance of Nafion to O₂ and CO, but not H₂, when bonded to precious metals is entirely unexpected. In fact, efforts at modifying molecular transport through Nafion have focused on increasing rather than decreasing this property for the purposes of cathodic oxygen reduction. The effect of phenolics to reduce molecular transport of Nafion on precious metals is unexpected.

### Example 3

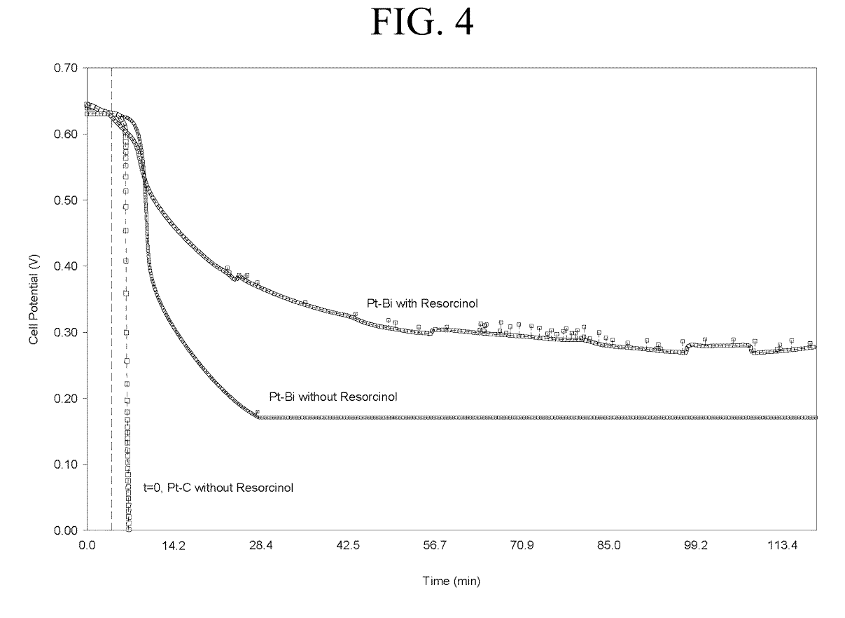

An experiment was conducted using same conditions as described in Example 1, except that an acetone-water mixture was employed as the liquid instead of an IPA-water mixture and the chronoamperometry measurement was conducted at 1 A (0.25 A/cm²). Referring to FIG. 4, the upper line shows the impact of adding resorcinol in increasing the power (voltage) relative to a PtBi catalyst alone without the resorcinol. Time zero indicates when the switchover from pure hydrogen to hydrogen containing 1% CO occurred. This coincides with the rapid drop-off to zero voltage the occurs with a typical pure Pt catalyst, with or without resorcinol.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. An electrocatalyst ink composition comprising:
a. a liquid vehicle;
b. particles comprising at least one electrocatalyst metal; and
c. at least one compound comprising a phenolic moiety.

2. The electrocatalyst ink of claim 1 wherein the compound comprising a phenolic moiety is selected from the group consisting of resorcinol, phenol, catechol, hydroquinone, phloroglucinol, cresol, halophenol, aminophenol, hydroxybenzoic acid, vannilic acid, vanillin, ferulic acid, benzoic acid, tiglic acid, and dihydroxybiphenyl.

3. The electrocatalyst ink of claim 1 wherein the compound comprising a phenolic moiety is resorcinol.

4. The electrocatalyst ink of claim 1 wherein the electrocatalyst metal is selected from the group consisting of carbon, palladium, ruthenium, rhodium, gold, copper, cobalt, nickel, and the rare earth elements.

5. The electrocatalyst ink of claim 1 wherein the electrocatalyst metal comprises a Platinum-Bismuth alloy.

6. The electrocatalyst ink of claim 1 wherein the particles are in a form selected from the group consisting of nanoparticles, pellets, platelets and combinations thereof.

7. The electrocatalyst ink of claim 1 wherein the particles are in the form of nanoparticles.

8. The electrocatalyst ink of claim 7 wherein the nanoparticles are attached to a support.

9. The electrocatalyst ink of claim 8 wherein the support comprises carbon.

10. A method for the fabrication of an electrocatalyst layer comprising depositing an ink composition onto a substrate, wherein said ink composition comprises a liquid vehicle, particles comprising at least one electrocatalyst metal, and at least one compound comprising a phenolic moiety.

11. The method of claim 10 wherein the substrate comprises a proton exchange polymer.

12. The method of claim 11 wherein the proton exchange polymer is Nafion.

13. The method of claim 10 wherein the compound comprising a phenolic moiety is selected from the group consisting of resorcinol, phenol, catechol, hydroquinone, phloroglucinol, cresol, halophenol, aminophenol, hydroxybenzoic acid, vannilic acid, vanillin, ferulic acid, benzoic acid, tiglic acid, and dihydroxybiphenyl.

14. The method of claim 10 wherein the compound comprising a phenolic moiety is resorcinol.

15. The method of claim 10 wherein the electrocatalyst metal is selected from the group consisting of carbon, palladium, ruthenium, rhodium, gold, copper, cobalt, nickel, and the rare earth elements.

16. The method of claim 10 wherein the electrocatalyst metal comprises a Platinum-Bismuth alloy.

17. The method of claim 10 wherein the particles are in a form selected from the group consisting of nanoparticles, pellets, platelets and combinations thereof.

18. The method of claim 10 wherein the particles are in the form of nanoparticles.

19. The method of claim 18 wherein the nanoparticles are attached to a support.

20. The method of claim 19 wherein the support comprises carbon.
